(19) United States
(12) Patent Application Publication
(10) Pub. No.: US 2025/0327817 A1
(43) Pub. Date: Oct. 23, 2025

(54) DETECTION OF PREDICTORS OF PREECLAMPSIA

(71) Applicant: UNIVERSITY OF IOWA RESEARCH FOUNDATION, Iowa City, IA (US)

(72) Inventors: Justin L. GROBE, Iowa City, IA (US); Mark K. SANTILLAN, Iowa City (IA); Donna SANTILLAN, Iowa City, IA (US)

(21) Appl. No.: 18/954,943

(22) Filed: Nov. 21, 2024

Related U.S. Application Data
(63) Continuation of application No. 16/603,952, filed on Oct. 9, 2019, now abandoned.
(60) Provisional application No. 62/484,050, filed on Apr. 11, 2017.

(51) Int. Cl. G01N 33/68

(52) U.S. Cl. CPC G01N 33/689; G01N 2800/368; G01N 2800/50

(57) ABSTRACT

This disclosure relates to improved detection of predictors of preeclampsia.

### BACKGROUND OF THE INVENTION

### Field of the Invention

This disclosure relates to improved detection of predictors of preeclampsia.

### Description of Related Art

Preeclampsia is a serious hypertensive disorder in pregnancy that can cause maternal complications including headaches, edema, liver and renal damage, seizures, and death. Women who experience preeclampsia during pregnancy are also at a greater life-long risk for cardiovascular diseases including hypertension, stroke, myocardial infarction, and cardiovascular death. According to the 2011 California Pregnancy Associated Mortality Review, a delay in the diagnosis of preeclampsia contributed to the cause of 92% of the maternal deaths in California among women with preeclampsia (see The California Pregnancy-Associated Mortality Review. Report from 2002 and 2003 Maternal Death Reviews. Sacramento, CA: California Department of Public Health, Maternal Child and Adolescent Health Division; 2011). Although detection of an early predictor of preeclampsia could potentially save lives, there are few simple or reliable methods currently available to predict which women will develop preeclampsia.

Recently, it was established that elevated maternal plasma copeptin, the pro-segment of arginine vasopressin, is highly predictive of the development of preeclampsia (see PCT/US2014/015627, PCT/US2014/015631, and Santillan M K, Santillan D A, Scroggins S M, Min J Y, Sandgren J A, Pearson N A, Leslie K K, Hunter S K, Zamba G K, Gibson-Corley K N, Grobe J L. Vasopressin in preeclampsia: A novel very early human pregnancy biomarker and clinically relevant mouse model. Hypertension, 2014, each of which is incorporated by reference in its entirety for all purposes). Development of preeclampsia could be predicted as early as the 6^{th} week of gestation despite the observation that clinical symptoms do not typically occur until after the 20^{th} to 24^{th} week of gestation with most cases developing in the late third trimester. The case-controlled study demonstrated that copeptin is robustly predictive of the development of preeclampsia in the 1^{st}, 2^{nd}, and 3^{rd} trimesters. Furthermore, clinically significant sensitivity, specificity, negative predictive value, and positive predictive value were demonstrated for copeptin even when controlling for significant confounders (Santillan et al.).

Apelin, a peptide hormone produced in magnocellular neurons of the hypothalamus, has been suggested to be useful for diagnosing preeclampsia. Arginine vasopressin (AVP) and apelin act in opposition, as AVP is known to increase blood pressure and increase water reabsorption, whereas apelin reduces blood pressure and increases diuresis (see Lee et al., Characterization of apelin, the ligand for the APJ receptor. J Neurochem, 2000. 74(1): 34-41; Reaux et al., Physiological role of a novel neuropeptide, apelin, and its receptor in the rat brain. J Neurochem, 2001. 77(4): 1085-96; Tatemoto et al., The novel peptide apelin lowers blood pressure via a nitric oxide-dependent mechanism. Regul Pept, 2001. 99(2-3): 87-92; and De Mota et al., Apelin, a potent diuretic neuropeptide counteracting vasopressin actions through inhibition of vasopressin neuron activity and vasopressin release. Proc Natl Acad Sci USA, 2004, 101(28): 10464-9). Application of apelin fragments such as K17F upon AVP-expressing neurons causes a reduction in firing rate (see DeMota et al.). Intracerebroventricular injection of K17F reduces plasma AVP levels and reduces the osmolality of urine (see DeMota et al.).

Interestingly, apelin has been reported to be elevated in the latter half (24-42 weeks) of pregnancies that have already developed the clinical symptoms of preeclampsia (see Simsek et al., Serum levels of apelin, salusin-alpha and salusin-beta in normal pregnancy and preeclampsia. J Matern Fetal Neonatal Med, 2012, 25(9): 1705-8; Inuzuka et al., Decreased expression of apelin in placentas from severe pre-eclampsia patients. Hypertens Pregnancy, 2013, 32(4): 410-21; and Kucur et al., Maternal serum apelin and YKL-40 levels in early and late-onset pre-eclampsia. Hypertens Pregnancy, 2014: 1-9). In contrast, others have reported that at delivery, apelin concentrations in maternal plasma are reduced in preeclamptic pregnancies (see Bortoff et al., Decreased maternal plasma apelin concentrations in preeclampsia. Hypertens Pregnancy, 2012, 31(4): 398-404). Yet, apelin levels do not appear to have been reported in early pregnancies that later develop preeclampsia.

An abnormal ratio of AVP to apelin has been proposed to contribute to water retention in patients with the Syndrome of Inappropriate Antidiuretic Hormone (SIADH) and chronic heart failure (see Blanchard et al., An abnormal apelin/vasopressin balance may contribute to water retention in patients with the syndrome of inappropriate antidiuretic hormone (SIADH) and heart failure. J Clin Endocrinol Metab, 2013, 98(5): 2084-9). Interestingly, inappropriately high apelin-to-copeptin ratios were present in these patients.

A second APJ ligand has recently been identified that may be relevant for development of preeclampsia. ELABELA (see, e.g., U.S. Pat. No. 9,309,314, incorporated herein by reference) is a 32 amino acid peptide hormone secreted by the placenta that is involved in AVP/copeptin release. Indeed, ELABELA knockout mice develop symptoms of preeclampsia (see Yi, et al. ELABELA deficiency promotes preeclampsia and cardiovascular malformations in mice. Science, 10.1126/science.aam6607 (2017)). However, one group showed that injection of ELABELA into the brain stimulated activity of AVP neurons, but did not show increased AVP release. (see, Santoso et al. Central action of ELABELA, NeuroReport, 2015, 26:820-826).

Ghrelin (also known as growth hormone-releasing peptide (GHRP)) is a 28 amino acid peptide hormone produced primarily in the stomach, but it is present in the central nervous system at low levels along with its receptor. Ghrelin has been shown to participate in growth hormone release, food intake, blood pressure regulation, and plays significant roles in energy metabolism. Ghrelin is also known to stimulate AVP/Copeptin release. Indeed, elevated blood ghrelin levels have been correlated with disease severity in pregnancies complicated by preeclampsia (see Erol et al. Increased serum ghrelin in preeclampsia: Is ghrelin a friend or a foe? Ginekologia Polska, 2016, 87, 277-282).

Because of the health risks associated with preeclampsia, early and reliable detection of predictors of preeclampsia are desirable. Early and accurate prediction of preeclampsia can be particularly important to enable early medical intervention and improved patient health. Therefore, there is a need for improved tools and methods for detecting predictors of preeclampsia.

### SUMMARY OF THE INVENTION

It is against the above background that the present invention provides certain advantages and advancements over the prior art. In particular, as set forth herein, early detection of apelin and copeptin is predictive of the development of preeclampsia.

In a first aspect, the invention provides a method of detecting predictors of preeclampsia in a human patient, the method includes: a. obtaining a sample from a pregnant patient; b. detecting a level of copeptin in the sample by applying the sample to a copeptin detection assay; and c. detecting a level of apelin or a fragment thereof in the sample by applying the sample to an apelin detection assay.

In one embodiment of the first aspect, the sample comprises at least one of whole blood, serum, plasma, or urine. In one embodiment of the first aspect, the bodily sample is a fresh sample or a frozen sample. In one embodiment of the first aspect, the copeptin detection assay comprises a test strip, an antibody detection assay, column chromatography, gas chromatography, or mass spectrometry. In one embodiment of the first aspect, the apelin detection assay comprises a test strip, an antibody detection assay, column chromatography, gas chromatography, or mass spectrometry. In one embodiment of the first aspect, the antibody detection assay comprises at least one of an ELISA, an immunoblot, and a radioimmunoassay. In one embodiment of the first aspect, the antibody detection assay comprises at least one antibody selected from a natural protein, a natural protein fragment, a synthetic protein, a synthetic protein fragment, or a nucleic acid. In one embodiment of the first aspect, the nucleic acid comprises an aptamer. In one embodiment of the first aspect, the antibody is specific for copeptin or a fragment thereof. In one embodiment of the first aspect, the antibody is specific for apelin or a fragment thereof. In one embodiment of the first aspect, the antibody is produced in response to antigenic stimuli of foreign proteins. In one embodiment of the first aspect, the sample was taken during the first trimester.

In a second aspect, the invention provides a method of detecting apelin and copeptin in a human patient, the method includes: a. obtaining a sample from the patient during the first trimester of pregnancy; b. detecting an elevated copeptin level in the sample compared to a control by contacting the sample with an antibody specific for copeptin; c. detecting binding between copeptin and the antibody specific for copeptin; d. detecting a depressed apelin level in the sample compared to a control by contacting the sample with an antibody specific for apelin; and e. detecting binding between apelin and the antibody specific for apelin.

In one embodiment of the second aspect, the elevated copeptin level and the depressed apelin level in combination is a predictor of the development of preeclampsia in the patient. In one embodiment of the second aspect, the sample comprises at least one of blood, serum, plasma, or urine. In one embodiment of the second aspect, the sample is taken during the sixth gestational week or earlier.

In a third aspect, the invention provides a method of treating preeclampsia in a human patient, the method includes: a. obtaining a sample from the patient during the first trimester; b. detecting an elevated level of copeptin in the sample by applying the sample to a copeptin detection assay; c. detecting a depressed level of apelin in the sample by applying the sample to an apelin detection assay; and d. administering a treatment to the patient for preeclampsia.

In one embodiment of the third aspect, the treatment comprises administering K17F or E339-3D6 to the patient. In one embodiment of the third aspect, the treatment comprises apheresis. In one embodiment of the third aspect, the treatment reduces copeptin levels in the patient. In one embodiment of the third aspect, the treatment increases apelin levels in the patient.

In a fourth aspect, the invention provides a method of detecting a plurality of preeclampsia predictive markers in a pregnant subject, the invention includes collecting a sample from the subject, detecting a first preeclampsia predictive marker in the sample, and detecting a second preeclampsia predictive marker in the sample.

In one embodiment of the fourth aspect, the first preeclampsia predictive marker and the second preeclampsia predictive marker are each selected from the group consisting of a vasopressin gene product, apelin, ELABELA, ghrelin, obestatin, soluble fms-like tyrosine kinase 1 (sFlt-1), endoglin, PLGF, SENG, LNPEP, ACE2, oxytocin, renin, an angiotensin gene product, and histones H3 and H4. The vasopressin gene product can be copeptin and/or neurophysin II. The angiotensin gene product can be one or more of angiotensin fragments I, II, III, IV, 1-9, 1-7, and 1-5. In one embodiment of the fourth aspect, the sample is taken during the first trimester. In another embodiment of the fourth aspect, the sample is taken after the first trimester.

In a fifth aspect, the invention provides a test device for predicting whether a subject is predisposed to developing preeclampsia, the device including a substrate comprising a test assay for detection of a protein product of the vasopressin gene; and a substrate comprising a test assay for detection of apelin, ELABELA, and/or ghrelin.

In one embodiment of the fifth aspect, the substrate comprises plastic, glass, metal, cellulosic material, a polymer, a cloth, and combinations thereof.

In another embodiment of the fifth aspect, the test device further includes user instructions for using the device and interpreting the information provided by the device.

In a seventh aspect, the invention provides a method of diagnosing or predicting the likelihood of occurrence of preeclampsia in a subject. The method includes measuring differences in ghrelin levels in a sample collected from a subject during the first trimester of pregnancy compared to a control. Ghrelin levels can be measured using an antibody detection assay. The sample can be blood, serum, plasma, or urine. In one embodiment of the seventh aspect, a decrease in ghrelin levels of about ⅕ fold compared to the control is predictive of the occurrence of preeclampsia during the subject's pregnancy.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings:

FIG. 1A shows maternal plasma apelin levels in subjects that have no history of preeclampsia (PreE), have had PreE, and currently have PreE.

FIG. 1B shows maternal plasma copeptin levels in subjects that have no history of PreE, have had PreE, and currently have PreE.

FIG. 2A shows maternal Plasma soluble fms-like tyrosine kinase-1 (sFLT1) levels in subjects that have no history of PreE, have had PreE, and currently have PreE.

FIG. 2B shows the ratio of maternal plasma apelin:copeptin levels in subjects that have no history of PreE, have had PreE, and currently have PreE.

FIG. 3 shows apelin:copeptin ratios from PreE samples compared to control. The results show that the apelin:copeptin ratio for PrE is significantly lower than control (P=0.0007).

FIG. 4 shows copeptin:apelin ratios from PreE samples compared to control.

FIG. 5 shows apelin X copeptin levels from PreE samples compared to control.

FIGS. 6A-6E show copeptin levels from PreE plasma samples (n=7) compared to control (n=13, 6A), apelin levels from PreE plasma samples compared to control (6B), osmolality of the maternal plasma samples levels from PreE samples compared to control (6C), apelin/copeptin levels from PreE samples compared to control, and apelin/copeptin*osmolality. Based on these data, it is believed that the ratios of the hormones can provide a more robust diagnostic test than either hormone measured individually.

FIG. 7 shows first trimester maternal urine copeptin concentrations (pg/mL) in control (n=101) and Preeclamptic (PreE, n=99) samples from the Harvard cohort. PreE samples showed more than 3-fold greater copeptin levels than control (P<0.05).

FIG. 8 shows first trimester maternal urine copeptin concentrations (pg/mL) in control (n=15) and Preeclamptic (PreE, n=15) samples from the GAPPS cohort. PreE samples showed nearly 3-fold greater copeptin levels than control (P<0.05).

FIG. 9 shows plasma ghrelin concentrations (pg/mL) from Preeclamptic samples (n=25) compared to control (n=36). Unexpectedly, ghrelin plasma concentration was significantly lower (by greater than about 20%) in preeclamptic women than control (P=0.0225).

### DETAILED DESCRIPTION OF THE INVENTION

All publications, patents, and patent applications cited herein are hereby expressly incorporated by reference for all purposes.

Before describing the present invention in detail, a number of terms will be defined. As used herein, the singular forms “a,” “an,” and “the” include plural referents unless the context clearly dictates otherwise. For example, reference to an “antibody” means one or more antibodies.

It is noted that terms like “preferably,” “commonly,” and “typically” when used herein are not utilized to limit the scope of the description or to imply that certain features are critical, essential, or even important to the structure or function of the claimed invention. Rather, these terms are merely intended to highlight alternative or additional features that can or cannot be utilized in a particular embodiment of the present invention.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “about” indicates ±10% of a given value.

As used herein, the term “bodily sample” or “patient sample” or “experimental sample,” or “sample” interchangeably refer to whole blood, blood fractions, including separately serum and/or plasma, urine, tissue, a biopsy, cells, and bodily fluids, including, for example, sweat and tears, and any combination thereof isolated from an individual. Such samples may be fresh, frozen, or otherwise stored.

As used herein, the term “preeclampsia predictive markers” refers to genes, DNA, RNA, proteins, hormones, and/or cellular metabolites for which expression levels can be correlated with development and/or severity of preeclampsia. Examples of preeclampsia predictive markers include all vasopressin gene products including copeptin and neurophysin II and apelin, ELABELA, and ghrelin gene products. Examples of additional preeclampsia predictive markers include soluble fms-like tyrosine kinase 1 (sFlt-1) and endoglin, which are anti-angiogenic markers that may predict the onset of preeclampsia 5 weeks and 9 to 12 weeks, respectively, to the onset of disease. Further examples of markers that may be used to predict preeclampsia include PLGF, sENG, LNPEP, ACE2, oxytocin, renin, angiotensin gene products such as angiotensin fragments I, II, III, IV, 1-9, 1-7, and 1-5, and histones H3 and H4.

As used herein, the term “apelin” refers to the apelin gene and any expressed RNA or proteins or subparts, such as fragments, thereof.

As used herein, the term “ELABELA” refers to the ELA gene and any expressed RNA or proteins or subparts, such as fragments, thereof.

As used herein, the term “ghrelin” refers to the ghrelin gene and any expressed RNA or proteins or subparts, such as fragments, thereof. The ghrelin gene encodes the ghrelin-obestatin preproprotein that is cleaved to yield two peptides, ghrelin and obestatin. Therefore, reference to the term “ghrelin” herein can also include reference to the obestatin peptide. Further, instances where ghrelin or fragments thereof are measured also contemplate measurement of the obestatin peptide or fragments thereof along with or separately from the ghrelin peptide. For example, “ghrelin” can refer to the ghrelin peptide alone, the ghrelin and obestatin peptides together either joined or cleaved apart, or the obestatin peptide alone for all purposes herein.

Furthermore, the measurement of levels of “preeclampsia predictive markers,” such as apelin or apelin fragments, ELABELA or ELABELA fragments, or ghrelin or ghrelin fragments (and others), either alone or in any combination, is predictive of the onset and/or severity of preeclampsia. Additionally, the ratio of apelin, ELABELA, and/or ghrelin to copeptin or fragments thereof and/or other by-products of the vasopressin gene is also predictive of a subject developing preeclampsia. Measurement of levels of “preeclampsia predictive markers” later during pregnancy (e.g., during the late first trimester, second trimester, or third trimester) can also be diagnostic of the disease.

The present invention is based, at least in part, on the discovery that apelin appears to modulate AVP secretion during normal pregnancy and may be dysfunctional in preeclamptic pregnancies. Further, the present invention is based on the concept that a ratio between apelin (or its fragments) and AVP (or copeptin or its fragments) represents a predictive ratio that is more sensitive and specific for preeclampsia than measures of AVP (or copeptin) or apelin (or its fragments) in isolation. Further, as apelin functions in opposition to AVP in the control of various physiological endpoints, it is hypothesized that delivery of apelin (or its fragments such as K17F, or receptor agonists such as E339-3D6) may represent a novel therapeutic approach to treat preeclampsia.

Similarly, ELABELA and ghrelin are also believed to be involved in AVP modulation in pregnancy and may be dysfunctional in and/or causative of preeclamptic pregnancies. Ratios between ELABELA and/or ghrelin (or their fragments) and AVP (or copeptin or its fragments) represent a predictive ratio that is more sensitive and specific for preeclampsia than measures of AVP (or copeptin) or ELABELA and/or ghrelin (or their fragments) in isolation. Moreover, measuring differences in ELABELA and/or ghrelin (or their fragments) alone or in any combination can be predictive and/or diagnostic of preeclampsia.

It is contemplated herein that assays and methods for detection of preeclampsia predictive markers, such as apelin, ELABELA, ghrelin, and/or copeptin, and others, can be combined and further coupled with additional assays for preeclampsia including measurement of osmolality and Doppler velocimetry measurements on at least one of a subject's uterine and/or umbilical arteries or other pertinent vasculature including, but not limited to, the middle cerebral artery and ductus venosus. Further, assays and methods for detection of preeclampsia predictive markers, such as apelin, ELABELA, ghrelin, and/or copeptin, as described herein, can also be combined with primary placental vessel flow measurements using other technologies such as CT or MRI for prediction or diagnosis of preeclampsia. It is further contemplated that additional assays may be combined with those disclosed herein, such as pregnancy tests, serum screening for aneuploidy, neural tube defects, and others known in the art. Thus, a single platform or device can be used to screen for multiple conditions that can affect the mother and/or the fetus.

Contemplated methods and kits for diagnosing or predicting the likelihood of occurrence of preeclampsia in a subject can include one or more antibody detection or other assays (test assays) specific for at least the detection of apelin, ELABELA, ghrelin, and/or copeptin, or subparts thereof (e.g., K17F), and combinations thereof, in a sample taken from the subject. The sample can be taken early in pregnancy from the subject, for example, in the first trimester of pregnancy for prediction of development or later in pregnancy for diagnosis of disease.

Samples contemplated in the present disclosure include whole blood, blood fractions, including serum and/or plasma, urine, tissues, cells, and bodily fluids, including, for example, sweat and tears, and any combination thereof. One preferred sample is plasma. Another preferred sample is serum. Another preferred sample is urine. In one embodiment, a kit includes an antibody detection assay that can be used with plasma, serum, and/or urine, in other words, any bodily sample may be used for the single assay.

While antibody-based detection assays are contemplated herein, additional test assays or detection assays such as apelin-specific assays, ELABELA-specific assays, ghrelin-specific assays, copeptin-specific assays, or other assays that are specific for the protein products of apelin, ELABELA, ghrelin, or vasopressin genes are also contemplated herein. For example, protein-and/or peptide-specific assays, enzyme activity assays (enzyme detection assays), immune-PCR-based techniques employing nucleic acid-linked antibodies that can be measured by quantitative PCR, epitope pull down via antibody-linked magnetic particles, including nanoparticles, or other selectable tag, mass spectrometry, and combinations thereof are contemplated herein. Kits contemplated herein can include positive and negative control samples, assay reagents, as well as instructions.

A contemplated assay can include a test strip, an ELISA, or other antibody-based or other target-specific assay, such as an enzyme activity assay where the presence of a targeted enzyme is detected by chromogenic means and the like due to enzyme activity. Test strips can be prepared in the conventional manner such as is described in U.S. Pat. Nos. 6,210,971 or 5,733,787 to Bayer Corporation (Elkhart, IN). It is contemplated that the test strips can couple attachment of the targeted epitope with the initiation of one or more of a chromogenic, fluorogenic, or luminescent reaction, as is known in the art, to indicate binding of the desired target. Further, a test strip can be characterized as an absorbent substrate capable of immobilizing metabolites bound to a layer of support material. Well-known solid phase supports can include paper, cellulose, fabrics made of synthetic resin, e.g. nylon or unwoven fabric. The absorbent material is typically bound to a layer of support material such as glass fiber or a synthetic polymer sheet to provide structural support. Other suitable solid phase supports are contemplated herein.

Additional assay formats contemplated for use include dipsticks (e.g., allowing dipping of the assay device into a test sample), urine tests (e.g., configured to allow an individual to urinate onto an assay device), finger prick with test strip or disk formats (e.g., similar to blood glucose and/or cholesterol assays), and other technologies. In one embodiment, assay formats can be designed for single use, at home testing by an individual. In another embodiment, assay formats can be multiplexed for replication within a testing format, such as a testing format that include two or more tests for repeat testing at the same time and averaging of results. In a further embodiment, contemplated assay formats can be multiplexed for testing samples from multiple individuals at the same time, such as, for example only, in a 96-well plate format, where up to 96 different samples can be tested at the same time. Different numbers of tests (i.e., repeats of the same test) are contemplated for each assay format.

In another embodiment, contemplated diagnostic platforms include measurement of copeptin (or fragments thereof), ELABELA (or fragments thereof), ghrelin (or fragments thereof), and apelin (or fragments thereof) levels from a bodily sample using flow cytometry, fluorescence, color change, tissue staining, quantitative PCR, densitometry, western blot, bio-barcode, and the like.

Further, two (or more, such as three or four) assays can be combined in a single assay device, such as, for example a pregnancy test that uses chromogenic or other means (for example, based on urine analysis or other sample). In this embodiment, in addition to the pregnancy test, one or more tests for prediction of preeclampsia (as described elsewhere herein) would be included. In this embodiment, a “positive” result for pregnancy (the subject is pregnant) can be indicated by a first indicium and a “positive” result for the preeclampsia test (indicating a predisposition for preeclampsia) can be indicated by a second indicium.

In another embodiment, a multiple test assay is contemplated that tests for pregnancy and/or multiple preeclampsia predictive markers. In this way, a greater specificity for prediction of preeclampsia accompanying pregnancy can be measured in a single test. For example, a single multiple test assay can measure one or more levels of copeptin (or fragments thereof), ELABELA (or fragments thereof), ghrelin (or fragments thereof), and apelin (or fragments thereof). In this way, the multiple test assay can provide information regarding each of copeptin (or fragments thereof), ELABELA (or fragments thereof), ghrelin (or fragments thereof), and apelin (or fragments thereof) at one time. It is further envisioned that the test assay can also indicate whether an individual is pregnant. It is contemplated that such tests can predict development of preeclampsia when administered to a pregnant patient early in pregnancy and can be diagnostic of preeclampsia when administered later in pregnancy after onset of preeclampsia.

Test assays can be incorporated into single use devices that can be purchased by the end user (for example, a woman seeking to know whether she is pregnant and/or at risk for preeclampsia). The test assay devices can be employed by application of urine, blood, or other some other sample to a single or multiple portions thereof, incubating the test assay for a prescribed period of time, and comparing the result to an interpretation key. Incubation times can be for about 1 minute, about 5 minutes, about 10 minutes, about 15 minutes, or about 1 hour, or shorter or longer. Interpretation keys or explanations can be associated with a package in which the test assay device was purchased, available electronically (for example, from a website or via electronic mail), or on the test assay device itself to allow interpretation of test results. Instructions and/or labels can also be associated with the test device (for example, attached to the test device) or included within a package containing the test device. In one embodiment, a kit with one or more test devices and instructions for use and/or interpretation of results from use is contemplated.

Apelin to copeptin ratios in a sample from a pregnant woman with no history of preeclampsia compared to control are predictive of the occurrence of preeclampsia in the woman at levels of at least about less than about 2, or less than about 1.5, or less than about 1, or less than about 0.8, or less than about 0.6, or less than about 0.4, or less than about 0.2. Similar ranges for apelin/copeptin* osmolality are contemplated.

ELABELA to copeptin ratios in a sample from a pregnant woman with no history of preeclampsia compared to control are predictive of the occurrence of preeclampsia in the woman at levels of at least about less than about 2, or less than about 1.5, or less than about 1, or less than about 0.8, or less than about 0.6, or less than about 0.4, or less than about 0.2.

Ghrelin to copeptin ratios in a sample from a pregnant woman with no history of preeclampsia compared to control are predictive of the occurrence of preeclampsia in the woman at levels of at least about less than about 2, or less than about 1.5, or less than about 1, or less than about 0.8, or less than about 0.6, or less than about 0.4, or less than about 0.2.

In another embodiment, a decrease of ghrelin levels in a sample taken during the first trimester from a pregnant subject compared to a control is predictive of the occurrence of preeclampsia during the subject's pregnancy. Decreases in ghrelin levels in a sample compared to control are considered to be predictive of the occurrence of preeclampsia during the subject's pregnancy including, for example, of about 1/100 fold, or about 1/50 fold. or about 1/25 fold, or about 1/10 fold, or about ⅕ fold, or greater or less.

In one embodiment, a method of diagnosing or predicting the likelihood of occurrence of preeclampsia in a subject can include collecting a sample, such as, urine, from the subject during the first trimester of pregnancy, measuring apelin (or an apelin fragment), ELABELA (or an ELABELA fragment), ghrelin (or a ghrelin fragment), and/or copeptin levels in the sample using, for example, an antibody detection assay or other assay, and determining whether the subject is likely to develop preeclampsia later in pregnancy by comparing the subject's apelin/copeptin, ELABELA/copeptin, and/or ghrelin/copeptin ratio levels to a control.

In another embodiment, a method of measuring the ratios of apelin:copeptin, ELABELA:copeptin, and/or ghrelin:copeptin in a sample from a pregnant subject includes collecting a sample from the subject during pregnancy, detecting a level of apelin (or an apelin fragment), ELABELA (or an ELABELA fragment), and/or ghrelin (or a ghrelin fragment), in the sample, detecting a level of copeptin in the sample, and measuring the ratio of apelin:copeptin, ELABELA:copeptin, and/or ghrelin:copeptin in the sample.

In a further embodiment, a method of measuring the ratio of apelin:copeptin, ELABELA:copeptin, and/or ghrelin:copeptin in a sample from a subject during the first trimester of pregnancy includes collecting a sample from the subject during the first trimester of pregnancy, detecting a level of apelin (or an apelin fragment), ELABELA (or an ELABELA fragment), and/or ghrelin (or a ghrelin fragment), in the sample, detecting a level of copeptin in the sample, and measuring the ratio of apelin:copeptin, ELABELA:copeptin, and/or ghrelin:copeptin in the sample.

In another embodiment, a method of diagnosing or predicting the likelihood of occurrence of preeclampsia in a subject can include collecting a sample, such as, urine, from the subject during the first trimester of pregnancy or later during pregnancy, measuring apelin (or an apelin fragment), ELABELA (or an ELABELA fragment), ghrelin (or a ghrelin fragment), and/or copeptin levels in the sample using, for example, an antibody detection assay or other assay, and determining whether the subject is likely to develop preeclampsia later in pregnancy or has preeclampsia by comparing the subject's apelin, ELABELA, ghrelin, and/or copeptin levels to a control.

In one embodiment, detection of a fragment of copeptin can be used to determine the level of copeptin in a sample. Similarly, detection of a fragment of apelin, ELABELA, or ghrelin can be used to determine the level of apelin, ELABELA, or ghrelin, respectively, in a sample.

In one embodiment, a method of detecting a plurality of preeclampsia predictive markers in a pregnant subject includes collecting a sample from the subject during pregnancy, detecting a first preeclampsia predictive marker in the sample, and detecting a second preeclampsia predictive marker in the sample.

In another embodiment, a method of detecting a plurality of preeclampsia predictive markers in a pregnant subject includes collecting a sample from the subject during the first trimester, second trimester, or third trimester of pregnancy, detecting a first preeclampsia predictive marker in the sample, and detecting a second preeclampsia predictive marker in the sample.

In another embodiment, a method for diagnosing preeclampsia includes measurement of one or more preeclampsia predictive markers in a pregnant subject during the first trimester and during the second and/or third trimester of pregnancy, where the preeclampsia predictive markers can be the same or different between trimesters, and comparing different levels of preeclampsia predictive markers between trimesters.

In one embodiment, it is believed that measurement of levels and/or ratios of preeclampsia predictive markers alone or in combination with other assays for preeclampsia as described herein in a non-pregnant subject (i.e., before pregnancy) can be predictive of the development and/or severity of preeclampsia during pregnancy. While not wishing to be bound by theory, it is believed, that at least in some women, preeclampsia might be a sub-clinical hypothalamic and/or renal hydromineral disorder that is unmasked when a woman becomes pregnant. In this context, specific contemplated methods of predicting development of preeclampsia during pregnancy in a patient before pregnancy include obtaining a sample from the patient and measuring: (1) urine, plasma, and/or serum copeptin; (2) urine, plasma, and/or serum osmolality; (3) urine, plasma, and/or serum levels of other preeclampsia predictive markers; or (4) any of the preceding in ratios to each other. Measured preeclampsia predictive marker levels and/or ratios can be compared to control, as described herein elsewhere.

In one embodiment, methods disclosed herein can further include measuring a ratio of first preeclampsia predictive marker to a second preeclampsia predictive marker.

Assays can provide data, for example, by color changes, light emission, changes in light emission intensity, densitometry, and/or changes in opacity/translucence of a substrate. These data, in turn, can be converted to data points that may be plotted compared to controls.

By early pregnancy, we mean at least before 20 weeks of amenorrhrea, more preferably, at least before about 16, or about 12, or about 8, or about 6 weeks, or about 4weeks of pregnancy. Early in pregnancy can also be during the first trimester.

By “patient” or “subject,” it is meant a female subject, such as, a human. Controls contemplated herein can comprise a single healthy pregnant age-matched subject, or a population of multiple healthy pregnant age-matched subject subjects or multiple healthy pregnant subjects, or serum and/or urine samples from a population of multiple healthy pregnant subjects none of whom later develop preeclampsia during pregnancy. Controls can further include a partially or fully purified apelin, ELABELA, ghrelin, and/or copeptin standard that is included in an assay in parallel with a patient sample for comparison. In addition, a predetermined control can also be a negative predetermined control. For example, a negative predetermined control comprises one or multiple subjects who developed preeclampsia during pregnancy. It is further contemplated that preeclampsia predictive marker levels in a patient sample can be normalized to a total protein value of the sample for analysis.

Antibody detection assays contemplated herein can include assays that use antibodies or antibody subparts to target a specific molecule of interest. Detection of the molecule can occur via antibody attachment to the molecule in combination with an indicator associated with the antibody or antibody subpart. It is further envisioned that the preeclampsia predictive markers of interest, such as, apelin, ELABELA, ghrelin, and/or copeptin, may be measured chromatographically, such as by column chromatography, gas chromatography, mass spectrometry, and combinations thereof. Examples of indicators to be attached to antibodies contemplated herein include various enzymes, a nucleic acid tag that can be used in immuno-PCR, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions, which can be conjugated to antibodies for use as diagnostics according to the present invention. Non-limiting examples of suitable enzymes include horseradish peroxidase, alkaline phosphatase, betagalactosidase, or acetylcholinesterase; non-limiting examples of suitable prosthetic group complexes include streptavidin/biotin and avidin/biotin; non-limiting examples of suitable fluorescent materials include umbelliferone, fluorescein, fluorescein isothiocyanate, rhodamine, dichlorotriazinylamine fluorescein, dansyl chloride, or phycoerythrin; a non-limiting example of a luminescent material includes luminol; non-limiting examples of bioluminescent materials include luciferase, luciferin, and aequorin; and non-limiting examples of suitable radioactive material include 125I, 131I, 111In, or 99Tc.

One example of an antibody detection assay is an ELISA. An ELISA can include antibodies specific for antigens or epitopes of preeclampsia predictive markers, such as apelin or a fragment thereof, ELABELA or a fragment thereof, ghrelin or a fragment thereof, and/or copeptin or a fragment thereof, or other coexpressed regions of the protein product of the vasopressin (AVP) gene, such as vasopressin and neurophysin II. An antigen can be a natural or synthetic protein or fragment thereof, polysaccharide, or nucleic acid. Skilled artisans know that antigens can induce an immune response and elicit antibody formation. Antibodies can be molecules synthesized in response to the presence of a foreign substance, wherein each antibody has specific affinity for the foreign material that stimulated its synthesis. The specific affinity of an antibody need not be for the entire molecular antigen, but for a particular site on it called the epitope (Kindt et al., Kuby Immunology, 6th Edition 574 pps, (2006)). Antibodies can be, for example, a natural or synthetic protein or fragment thereof or nucleic acids (e.g., aptamers) with protein-binding or other antigen-binding characteristics. Antibodies can be produced in response to antigenic stimuli including, but not limited to, exposure to foreign proteins, microorganisms, and toxins. One of ordinary skill in the art can assess antigen-antibody immunocomplex formation by techniques commonly used in the art. Examples of suitable additional assays to assess immunocomplex formation contemplated herein include phage immunoblot and radioimmunoassay. See, e.g., (Dubovsky et al., J. Immunother. 30:675-683 (2007).

In one embodiment, a non-limiting example of an apelin fragment is K17F. K17F is defined here as having the amino acid sequence Lys-Phe-Arg-Arg-Gin-Arg-Pro-Arg-Leu-Ser-His-Lys-Gly-Pro-Met-Pro-Phe.

In another embodiment, methods for treating preeclampsia are contemplated. For example, preeclampsia can be treated by the delivery of an effective amount of apelin, an apelin fragment, or an apelin receptor agonist, such as E339-3D6, which would increase apelin concentrations to normal levels. Similarly, ghrelin may be administered to a patient to return ghrelin plasma levels to normal to treat preeclampsia.

In another embodiment which employs a procedure similar to dialysis referred to as apheresis, it is contemplated to sample blood from a patient by passing the patient's blood through a column or functionally similar device that captures apelin, ELABELA, ghrelin, copeptin, or vasopressin and allows the blood to return to the patient to allow apelin, ELABELA, ghrelin, and copeptin levels to be measured. It is further contemplated to use dialysis. In another embodiment, when indicated, it is contemplated that the process can be used to increase (by adding) or reduce (by capturing) apelin, ELABELA, ghrelin, copeptin, and/or vasopressin levels in the patient's blood stream, as required. A similar system can be used to test waste samples from a patient for elevated apelin or copeptin levels, for example, where a urine sample is passed through a column or similar device packed with a medium to which are attached anti-apelin-, anti-ELABELA-, anti-ghrelin-, or anti-copeptin antibodies and/or similar apelin, ELABELA, ghrelin, and/or copeptin-specific binding agent(s).

In a further embodiment, contemplated tests for early prediction of preeclampsia can combine measurement of preeclampsia predictive marker levels, such as apelin, ELABELA, ghrelin, and/or copeptin levels with one or more of cell-free fetal DNA, cell-free total DNA, and pregnancy-associated plasma protein A levels.

In one embodiment, methods of predicting preeclampsia in a pregnant woman can include combining a bodily sample from the woman with an assay solution. The assay solution can include buffers, saline, antigen-binding agents (e.g., second preeclampsia predictive marker, such as copeptin-binding agents, apelin-binding agents, ELABELA-binding agents, ghrelin-binding agents, or other antigen specific binding agents). nucleotides, salts, nucleic acid primers, DNA polymerases, fluorescent compounds, and combinations thereof, which enable quantification of antigen or protein found within the sample. In some embodiments, an apelin-specific binding agent, an ELABELA-specific binding agent, ghrelin-specific binding agent, and/or a copeptin-specific binding agent is included in the assay solution, which is adapted to complex with apelin. ELABELA, ghrelin, and/or copeptin, respectively, in the solution to form an assay mixture comprising apelin-binding agent, ELABELA-binding agent, ghrelin-binding agent, and copeptin-binding agent complexes. The assay mixture can be assayed subsequently to measure the amount of apelin, ELABELA, ghrelin, or copeptin and/or number of apelin-binding, ELABELA-binding, ghrelin-binding, or copeptin-binding agents. As an alternative, the bodily sample can be first applied to a substrate of an assay and an assay may be subsequently added to the bodily sample.

In certain embodiments, anti-preeclampsia predictive marker antibodies, such as anti-copeptin, anti-ELABELA, anti-ghrelin, or anti-apelin antibodies, or antigen binding fragments thereof, can be chimerized, humanized, or deimmunized. In one embodiment, an antibody, or antigen binding fragments thereof, of the invention can be chimeric. A chimeric antibody is an antibody in which different portions of the antibody are derived from different animal species, such as antibodies having a variable region derived from a murine monoclonal antibody and a human immunoglobulin constant region. Methods for producing chimeric antibodies, or fragments thereof, are known in the art. See e.g., Morrison, Science 229:1202, 1985; Oi et al., BioTechniques 4:214, 1986; Gillies et al., J. Immunol. Methods 125:191, 1989; U.S. Pat. Nos. 5,807,715; 4,816,567; and 4,816,397. Techniques developed for the production of “chimeric antibodies” (Morrison et al., Proc. Natl. Acad. Sci. 81:851, 1984; Neuberger et al., Nature 312:604, 1984; Takeda et al., Nature 314:452, 1985) can be employed for the synthesis of the molecules. For example, a genetic sequence encoding a binding specificity of a mouse anti-copeptin, anti-ELABELA, anti-ghrelin, or anti-apelin antibody molecule can be fused together with a sequence from a human antibody molecule of appropriate biological activity. As used herein, a chimeric antibody is a molecule in which different portions are derived from different animal species, such as those having a variable region derived from a murine monoclonal antibody and a human immunoglobulin constant region, e.g., humanized antibodies.

In another embodiment, an antibody, or antigen-binding fragment thereof, of the invention is humanized. Humanized antibodies have a binding specificity comprising one or more complementarity determining regions (CDRs) from a non-human antibody and framework regions from a human antibody molecule. Often, framework residues in the human framework regions will be substituted with the corresponding residue from the CDR donor antibody to alter, preferably improve, antigen binding. These framework substitutions are identified by methods well known in the art, e.g., by modelling of the interactions of the CDR and framework residues to identify framework residues important for antigen binding and sequence comparison to identify unusual framework residues at particular positions. See e.g. Queen et al., U.S. Pat. No. 5,585,089; Riechmann et al., Nature 332:323, 1988. Antibodies can be humanized using a variety of techniques known in the art including, for example, CDR-grafting (EP 239,400; International Publication No. WO 91/09967; U.S. Pat. Nos. 5,225,539; 5,530,101; and 5,585,089), veneering or resurfacing (EP 592,106; EP 519,596; Padlan, Molecular Immunology 28:489, 1991; Studnicka et al., Protein Engineering 7:805, 1994; Roguska. et al., PNAS 91:969, 1994), and chain shuffling (U.S. Pat. No. 5,565,332).

In some embodiments, an assay for prediction of preeclampsia, rather than taking a sample from a patient, includes introducing an anti-copeptin antibody, an anti-ELABELA antibody, an anti-ghrelin antibody, and/or an anti-apelin antibody into the patient and measuring copeptin, ELABELA, ghrelin, and/or apelin levels, respectively, in situ. Similarly, antibodies to other preeclampsia predictive markers can be used. In such embodiments, de-immunization can be used to decrease the immunogenicity of the antibody or antigen binding fragment thereof. As used herein, the term “de-immunization” includes alteration of an antibody, or antigen-binding fragment thereof, to modify T cell epitopes (see, e.g., International Publication Nos. WO9852976A1, WO0034317A2). For example, VH and VL sequences from the starting antibody can be analyzed and a human T cell epitope “map” can be generated from each variable region showing the location of epitopes in relation to complementarity-determining regions (CDRs) and other key residues within the sequence. Individual T cell epitopes from the T cell epitope map can be analyzed in order to identify alternative amino acid substitutions with a low risk of altering activity of the final antibody. A range of alternative VH and VL sequences may be designed comprising combinations of amino acid substitutions and these sequences may be subsequently incorporated into a range of copeptin-specific antibodies or fragments thereof for use in the diagnostic methods disclosed herein, which are then tested for function. Typically, between 12 and 24 variant antibodies may be generated and tested.

### EXAMPLES

The Examples that follow are illustrative of specific embodiments of the invention, and various uses thereof. They are set forth for explanatory purposes only, and are not to be taken as limiting the invention.

### Example No. 1. Measurement of Apelin:Copeptin Ratios

### Overview

Preeclampsia annually kills 76,000 mothers and 500,000 babies worldwide often due to delay in diagnosis secondary to the lack of simple, early gestation tests. Altered levels of apelin and circulating copeptin (CPP), the pro-segment of vasopressin, are associated with preeclampsia. It was previously demonstrated that CPP is robustly predictive of preeclampsia as early as the 6th week of gestation in all mothers, while altered apelin levels have been diagnostic of preeclampsia in the latter half of pregnancy. However, measuring apelin levels has not been considered useful as a predictor of development of preeclampsia nor have levels of apelin been reported in early pregnancy. Moreover, ratios of apelin to CPP have not been considered as predictive of the development of preeclampsia in pregnant women.

Here, maternal plasma was evaluated to determine whether apelin and/or ratios of apelin to CPP are predictive of preeclampsia.

### Materials and Methods:

Maternal blood was collected from pregnant patients into ACD-A tubes under the Maternal-Fetal Tissue Bank Institutional Review Board approved protocol (IRB#200910784). Blood was processed and plasma was snap-frozen and stored at −80° C. For these assays, third trimester samples were identified from pregnant women who 1) never had a diagnosis of preeclampsia (previous or current pregnancy) (n=2), 2) who had preeclampsia in a previous pregnancy (but not in the current pregnancy) (n=2), and 3) who had a diagnosis of preeclampsia in the current pregnancy (n=4). Samples were thawed on wet ice, brought to room temperature, and vortexed prior to use in the apelin assay. Apelin was measured using the Sigma-Aldrich EIA kit (catalog #RAB0018-1KT) according to the manufacturer's protocol. This kit is designed to target the C-terminus of the 77 amino acid apelin peptide, and therefore, is expected to detect all active apelin peptides, including Apelin-13, Apelin-28, Apelin-31, and Apelin-36. Samples were diluted 4 fold as described in the manufacturer's protocol. Each sample was measured in duplicate, and the average was taken. Absorbance was read at 450 nm using a BioRad xMark plate reader.

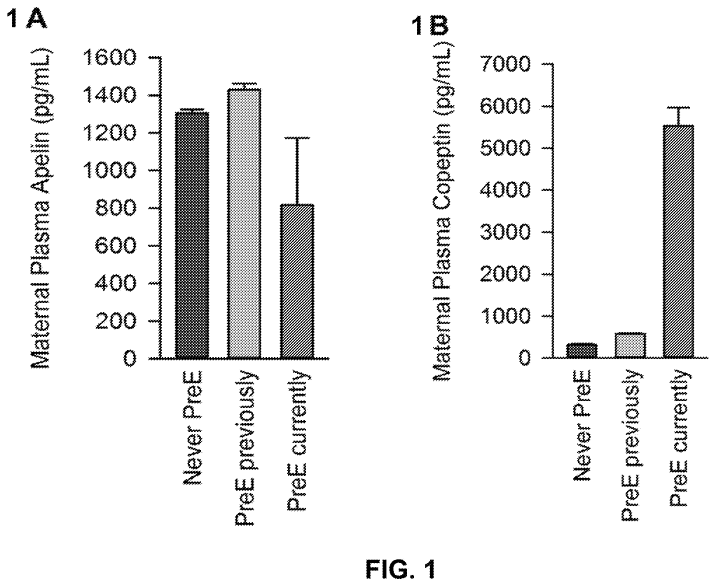

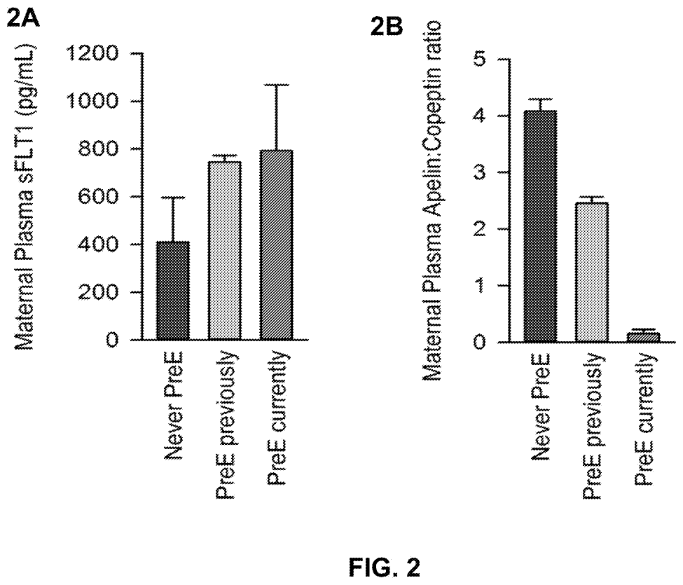

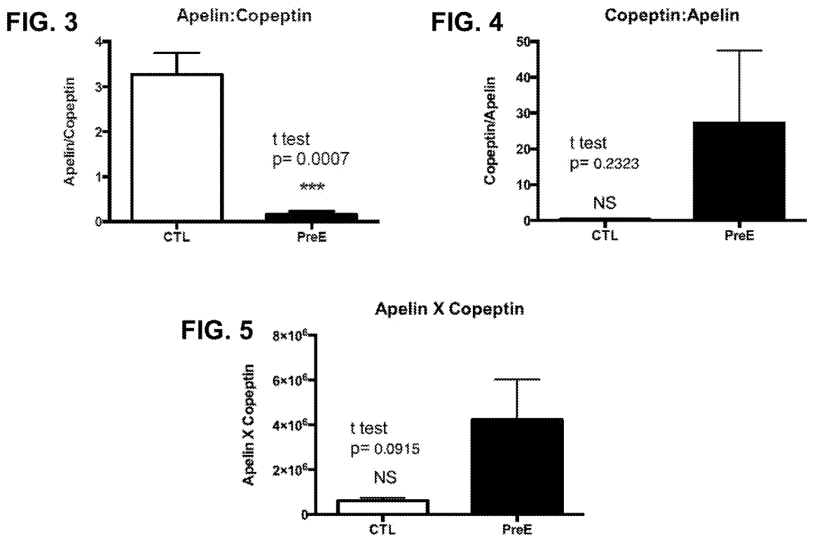

Results are shown in FIGS. 1-5.

### Example No. 2. Measurement of Apelin:Copeptin Ratios in Early Pregnancy

### Overview

The earlier preeclampsia can be accurately predicted in a pregnant woman, the more likely the woman can be treated to alleviate risk to her health and fetus in later pregnancy. Here, the ability of apelin:copeptin ratios measured in maternal blood samples taken from early in pregnancy for prediction of the development of preeclampsia was determined.

### Materials and Methods:

Maternal blood was collected from pregnant patients into ACD-A tubes under the Maternal-Fetal Tissue Bank Institutional Review Board approved protocol (IRB#200910784). Blood was processed and plasma was snap-frozen and stored at −80° C. For these assays, first trimester samples (from 6-11 weeks) were identified from pregnant women who 1) never had a diagnosis of preeclampsia (previous or current pregnancy) (n=13), and 2) who had a diagnosis of preeclampsia in the current pregnancy (n=7). Samples were treated and measured as described in Example No. 1.

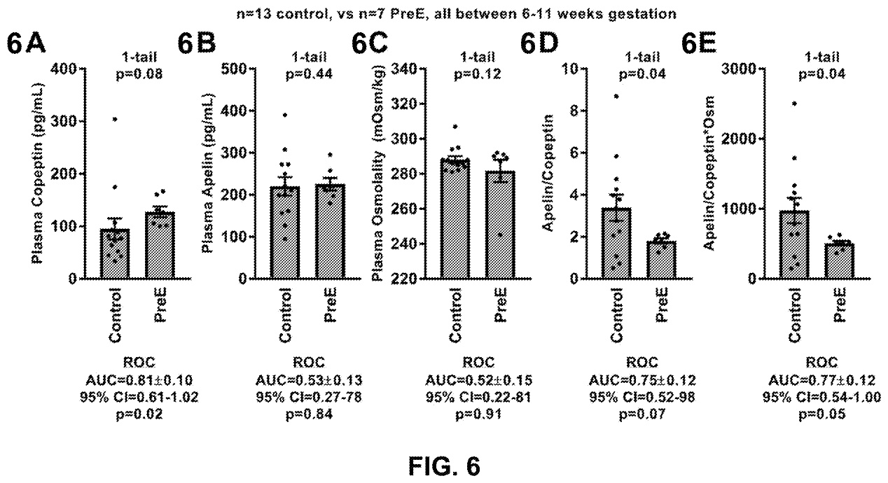

A further analysis was performed to determine the relationship between copeptin, apelin, and osmolality ratios as follows: apelin/copeptin, and apelin/copeptin*osmolality. Results are shown in FIGS. 6A-6E. In this cohort, plasma samples from first-trimester pregnant women who subsequently were diagnosed with preeclampsia (PreE), or case control subjects (controls), were analyzed for copeptin and apelin using ELISA assays and osmolality using freezing point depression osmometry. Similar to our previous publication (Santillan et al., Hypertension, 2014. 64(4): p. 852-9) and recent data from Jadli et al. (Placenta, 2017. 58: p. 67-73), first-trimester maternal plasma copeptin levels are elevated before the development of preeclampsia (FIG. 6A). Plasma apelin concentrations (FIG. 6B) and plasma osmolalities (FIG. 6C) do not appear to be different between preeclamptic and control subjects in the first trimester. Calculating the ratio between plasma apelin concentrations and plasma copeptin, either as a simple ratio (FIG. 6D), or as a ratio multiplied by the plasma osmolality (FIG. 6E), results in a much tighter clustering of the preeclampsia samples, and therefore improved analytical statistic outcomes. These data lead to the proposition that calculating the ratio or product of known biological regulators of vasopressin/copeptin secretion (such as apelin, ELABELA, ghrelin, osmolality, volume, etc.) versus copeptin (or vasopressin) will provide superior testing characteristics to predict the development of preeclampsia and associated pregnancy-related cardiovascular disease.

### Example No. 3. Measurement of Copeptin in Urine

### Overview

In this example, the ability of copeptin to be measured in maternal urine samples taken from early in pregnancy for prediction of the development of preeclampsia is demonstrated.

### Materials and Methods:

Maternal urine was collected from pregnant patients into specimen cups in clinic under the Maternal-Fetal Tissue Bank Institutional Review Board approved protocol (IRB#200910784).

Samples were poured into smaller tubes, snap frozen, and stored at −80° C. Urine was thawed on wet ice and brought to room temperature for use in a copeptin ELISA by USCN according to the manufacturer's protocol. Samples were assayed neat (no dilution) in duplicate and absorbances were averaged and used to calculate the sample concentration.

All urine samples were further normalized based on total protein content as determined using a bicinchonic acid assay (BCA) (Pierce catalog #23225). Samples were also normalized for osmolality.

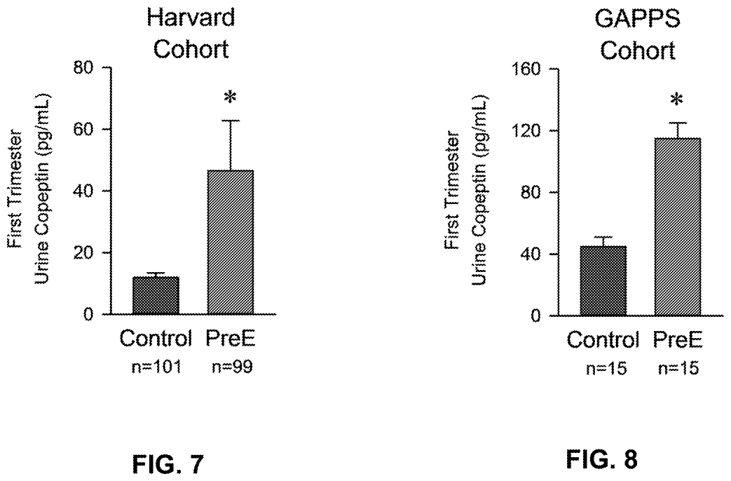

Results for copeptin urine levels are shown in FIGS. 7 and 8.

### Example No. 4. Measurement of Apelin-Copeptin Ratios in Urine

### Overview

In this example, the ability of apelin:copeptin ratios measured in maternal urine samples taken from early in pregnancy for prediction of the development of preeclampsia is determined.

### Materials and Methods:

Maternal urine was collected from pregnant patients into specimen cups in clinic under the Maternal-Fetal Tissue Bank Institutional Review Board approved protocol (IRB#200910784).

Samples are poured into smaller tubes, snap frozen, and stored at −80° C. Urine is thawed on wet ice and brought to room temperature for use in the Sigma-Aldrich Apelin EIA kit (catalog #RAB0018-1KT) according to the manufacturer's protocol, urine copeptin levels are measured as described in Example No. 3.

All urine samples are further normalized based on total protein content as determined using a bicinchonic acid assay (BCA) (Pierce catalog #23225). Samples are also normalized for osmolality.

Apelin:copeptin ratios are calculated.

### Example No. 5. Measurement of Ghrelin:Copeptin Ratios in Early Pregnancy

### Overview

The earlier preeclampsia can be accurately predicted in a pregnant woman, the more likely the woman can be treated to alleviate risk to her health and fetus in later pregnancy. Ghrelin is an upstream regulator of copeptin release. Therefore, increased expression of ghrelin may be predictive of preeclampsia in conjunction with or alone from copeptin. Here, the ability of ghrelin:copeptin ratios measured in maternal blood samples taken from early in pregnancy for prediction of the development of preeclampsia was determined. Identification of additional early biomarkers or diagnostic tests for preeclampsia could improve the outcomes of the pregnancy for the mother and child as well as lead to the development of novel therapeutics.

### Materials and Methods:

Maternal plasma was collected from pregnant patients into ACD-A tubes under the Maternal-Fetal Tissue Bank Institutional Review Board approved protocol (IRB#200910784). Plasma was processed and snap-frozen and stored at −80° C. For these assays, first trimester samples (from 6-11 weeks) were identified from pregnant women who 1) never had a diagnosis of preeclampsia (previous or current pregnancy) (n=36), and 2) who had a diagnosis of preeclampsia in the current pregnancy (n=25). A second cohort was tested that included preeclamptic (n=51) and control (n=320) patients. All samples were from the first trimester of pregnancy. Ghrelin plasma levels were measured using an anti-ghrelin ELISA kit (Platinum ELISA, eBioscience. Equal volumes of plasma were utilized for each sample. Each sample was analyzed in duplicate and the average values were obtained.

### Results and Discussion

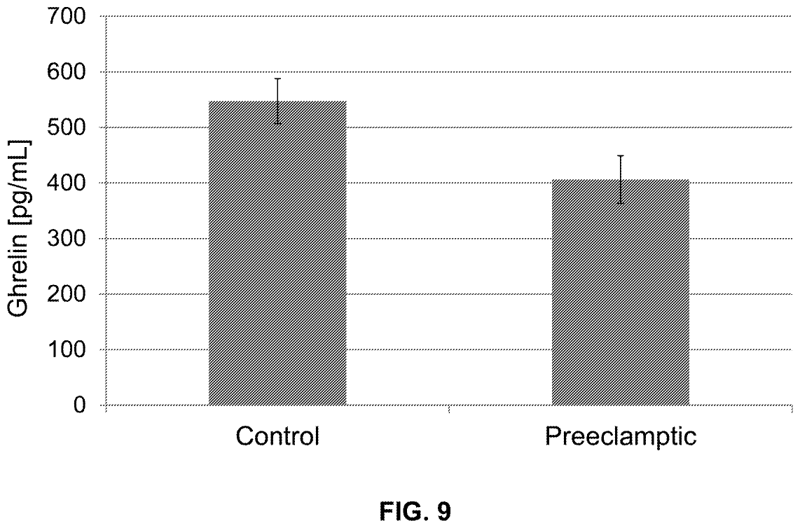

Results from the first cohort are shown in FIG. 9. Results from the second cohort are shown in Table No. 1 below.

TABLE NO. 1
Plasma ghrelin levels
Dependent Variable: Ghrelin (units)
Normality Test (Shapiro-Wilk): Failed (P < 0.050)
Equal Variance Test (Brown-Forsythe): Passed (P = 0.467)

| Group Name | N | Missing | Mean | Std Dev | SEM |
|---|---|---|---|---|---|
| 0.000 | 320 | 284 | 547.282 | 242.805 | 40.468 |
| 1.000 | 51 | 26 | 406.090 | 214.003 | 42.801 |

Difference 141.192
t = 2.342 with 59 degrees of freedom.
95 percent two-tailed confidence interval for difference of means: 20.583
to 261.802
Two-tailed P-value = 0.0225
The difference in the mean values of the two groups is greater than would
be expected by chance; there is a statistically significant difference
between the input groups (P = 0.023).
One-tailed P-value = 0.0113
The sample mean of group 0.000 exceeds the sample mean of group 1.000
by an amount that is greater than would be expected by chance, rejecting
the hypothesis that the population mean of group 1.000 is greater than or
equal to the population mean of group 0.000. (P = 0.011).
Power of performed two-tailed test with alpha = 0.050: 0.635
Power of performed one-tailed test with alpha = 0.050: 0.749

Ghrelin is known to stimulate AVP/copeptin release. However, our data demonstrate that increased first trimester copeptin, as a marker of vasopressin release, is a robust early predictor of the diagnosis of preeclampsia. Contrary to our expectation, these data demonstrate that first trimester ghrelin is significantly decreased in those who developed preeclampsia. Given that ghrelin is upstream and stimulates AVP/copeptin release, it is believed that the ratio of ghrelin to copeptin will improve the prediction characteristics of copeptin alone for the prediction of preeclampsia. Further, these data demonstrate (conversely to copeptin levels) that a significant decrease in ghrelin levels in the first trimester of pregnancy alone can be predictive of development of preeclampsia later in pregnancy.

### Example No. 6. Measurement of ELABELA:Copeptin Ratios in Early pregnancy

### Overview

Preeclampsia is a cardiovascular disorder of late pregnancy for which there are currently no effective diagnostic/predictive tests. We recently demonstrated that copeptin, a fragment of the vasopressin (AVP) gene, is grossly elevated in maternal blood already in very early pregnancy in those women who eventually develop preeclampsia. The cause of elevated AVP/copeptin secretion in preeclampsia remains unknown. Apelin is a potential regulator through its actions at the APJ receptor. ELABELA is a newly discovered hormone that acts through the APJ receptor and may contribute to AVP/copeptin release. ELABELA knockout in mice is sufficient to cause the mice to develop symptoms of preeclampsia. While the ratio of copeptin:apelin is predictive of preeclampsia for patent consideration (see above), the utility of copeptin:ELABELA ratios remains to be elucidated. Because ELABELA is important in development earlier than apelin, this new ratio (copeptin:ELABELA) may be more useful earlier in gestation to predict preeclampsia.

Here, maternal plasma is evaluated to determine whether ELABELA and/or ratios of ELABELA to CPP are predictive of preeclampsia. It is further contemplated that ratios of ELABELA protein or RNA in maternal plasma or urine, or combinations of polymorphisms in the copeptin or ELABELA or APJ genes in mother, father or fetus, may be predictive of the development of preeclampsia.

### Materials and Methods:

Maternal blood was collected from pregnant patients into ACD-A tubes under the Maternal-Fetal Tissue Bank Institutional Review Board approved protocol (IRB#200910784). Blood was processed and plasma was snap-frozen and stored at −80° C.

For these assays, third trimester samples are identified from pregnant women who 1) never had a diagnosis of preeclampsia (previous or current pregnancy), 2) who had preeclampsia in a previous pregnancy (but not in the current pregnancy), and 3) who had a diagnosis of preeclampsia in the current pregnancy. Samples are thawed on wet ice, brought to room temperature, and vortexed prior to use in the ELABELA assay. ELABELA is measured using an ELISA kit according to the manufacturer's protocol. Each sample is measured in duplicate, and the average is taken. Absorbance is read at 450 nm using a BioRad xMark plate reader. Copeptin levels are measured, as previously described above, and copeptin:ELABELA ratios are calculated and the results analyzed between groups.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention. Percentages disclosed herein may otherwise vary in amount by ±10, 20, or 30% from values disclosed herein.

1-16. (canceled)

17. A method of treating preeclampsia in a human patient, comprising:
a. obtaining a sample from a human patient during the first trimester;
b. detecting an elevated level of copeptin in the sample compared to control by applying the sample to a copeptin detection assay;
c. detecting a depressed level of apelin in the sample compared to control by applying the sample to an apelin detection assay; and
d. administering a treatment to the patient for preeclampsia.

18. The method of claim 17, wherein the treatment comprises administering apelin, ghrelin, K17F, and/or E339-3D6 to the patient.

19. The method of claim 17, wherein the treatment comprises apheresis.

20. The method of claim 19, wherein the treatment reduces copeptin levels in the patient.

21. The method of claim 18, wherein the treatment increases apelin levels in the patient.

22. (canceled)

23. The method of claim 17 further comprising detecting a level of one or more of neurophysin II, ELABELA, ghrelin, obestatin, soluble fms-like tyrosine kinase 1 (sFlt-1), endoglin, PLGF, sENG, LNPEP, ACE2, oxytocin, renin, an angiotensin gene product, and histones H3 and H4 in the sample.

24. (canceled)

25. The method claim 23, wherein the angiotensin gene product is one or more of angiotensin fragments I, II, III, IV, 1-9, 1-7, and 1-5.

26. The method of claim 17, wherein the sample is taken during the sixth gestational week or earlier.

27-31. (canceled)

32. The method of claim 17, wherein the sample comprises at least one of whole blood, serum, plasma, or urine.

33. The method of claim 32, wherein the bodily sample is a fresh sample or a frozen sample.

34. The method of claim 17, wherein the copeptin detection assay comprises a test strip, an antibody detection assay, column chromatography, gas chromatography, or mass spectrometry.

35. The method of claim 34, wherein the antibody detection assay comprises at least one of an ELISA, an immunoblot, and a radioimmunoassay.

36. The method of claim 35, wherein the antibody detection assay comprises at least one antibody selected from a natural protein, a natural protein fragment, a synthetic protein, and a synthetic protein fragment, or a nucleic acid.

37. The method of claim 36, wherein the nucleic acid comprises an aptamer.

38. The method of claim 36, wherein the antibody is specific for copeptin or a fragment thereof.

39. The method of claim 17, wherein the apelin detection assay comprises a test strip, an antibody detection assay, column chromatography, gas chromatography, or mass spectrometry.

40. The method of claim 39, wherein the antibody detection assay comprises at least one of an ELISA, an immunoblot, and a radioimmunoassay.

41. The method of claim 40, wherein the antibody detection assay comprises at least one antibody selected from a natural protein, a natural protein fragment, a synthetic protein, and a synthetic protein fragment, or a nucleic acid.

42. The method of claim 41, wherein the nucleic acid comprises an aptamer.

43. The method of claim 41, wherein the antibody is specific for apelin or a fragment thereof.
